(19) United States
(12) Patent Application Publication
(10) Pub. No.: US 2021/0161190 A1
(43) Pub. Date: Jun. 3, 2021

(54) CORE BODY TEMPERATURE REDUCING AGENT, FOOD PRODUCT, AND SLEEP IMPROVING AGENT

(71) Applicant: JAPAN TOBACCO INC., Tokyo (JP)

(72) Inventors: Yutaka KUROKI, Tokyo (JP); Koyomi MIYAZAKI, Tsukuba-shi (JP)

(73) Assignee: JAPAN TOBACCO INC., Tokyo (JP)

(21) Appl. No.: 17/268,129

(22) PCT Filed: Aug. 21, 2019
(86) PCT No.: PCT/JP2019/032688

§ 371 Date: Feb. 12, 2021

(30) Foreign Application Priority Data
Aug. 21, 2018 (JP) 2018-154827

(51) Int. Cl. A23L 33/105; A23L 2/52

(52) U.S. Cl. CPC A23L 33/105; A23V 2250/21; A23L 2/52

(57) ABSTRACT

Provided is a novel core body temperature reducing agent. The core body temperature reducing agent of the present invention includes an extract from Tongkat Ali as an active ingredient.

### TECHNICAL FIELD

The present invention relates to a core body temperature reducing agent, a food product including the core body temperature reducing agent, and a sleep improving agent including the core body temperature reducing agent.

### BACKGROUND ART

Most living things, including animals and plants, possess a physiological phenomenon that varies on an approximately 24-hour cycle, which is called circadian cycles (circadian rhythms). Additionally, homoiotherms, including humans, possess as one of the circadian rhythms a sleep-wake rhythm, i.e., a body temperature rhythm with a 24-hour cycle where body temperature is high during activity period and is low during rest period.

However, it is known that poor living habits, such as irregular lifestyles, poor eating habits, and lack of exercise disturb the 24-hour cycle rhythm, which causes variations in body temperature, and affects psychologically and physiologically. For example, sustained body temperature reduction during activity period (during awakening) is known as so-called “cold sensitivity”. There is also a suggestion that body temperature reduction is associated with physiological dysfunctions, such as weakened immune system, delayed wound healing, sleep disorder, obesity, and depression.

In addition, persistent high body temperature is referred to as so-called “heat retention”, which is caused because heat generated in the body cannot be well dissipated from the skin surface and is retained in the body. Examples thereof include heatstroke and thermal fever. If the condition becomes worse, reduced organ blood flow due to the increased body temperature and multiple organ failure may occur, leading to death. Even if not fatal, high body temperature situation is dangerous to life, because, for example, especially in severe cases, sequelae such as brain dysfunction and kidney disorder can occur. It is also known that core body temperature increases during exercise, and persistent increase in the core body temperature reduces exercise duration time. Thus, to exercise efficiently, it is necessary to moderately reduce core body temperature. Accordingly, maintaining an appropriate core body temperature is a critical matter for living organisms.

Recent years have seen an increasing number of people suffering from sleeping problems such as insomnia due to stress, depression, and the like. A large number of reports have been issued on methods and agents for improving sleep quality (content). However, in some of sleeping agents used as so-called medicines, there have been reported side effects, reduced effect due to continuous administration, and the like. Due to that, there is a need for a method that can induce sleep or provide continuous sleep in a way as natural as possible. Under such circumstances, attention is being paid to improvement of sleep quality (content) by normalization of internal core body temperature.

Sleep is categorized into REM sleep (sleep with rapid eye movement) and non-REM sleep (sleep without rapid eye movement, which is also referred to as slow-wave sleep), and non-REM sleep is referred to as so-called deep sleep. It is known that particularly, non-REM sleep is correlated with core body temperature. For example, NPL 1 describes a relationship between core body temperature and sleep, and states that reduced core body temperature induces sleep. In NPL 2, it is described that intake of an amino acid “glycine” reduces core body temperature, which improves the percentage of Non-REM sleep, resulting in improved quality (content) and amount of sleep.

Furthermore, it is also known that aging is greatly influential on the relationship between sleep and core body temperature. Elderly people are more prone to sleep disorders such as early-morning awakening and frequent night awakening. This is thought to occur because, in comparison with younger people, elderly people have less reduction in core body temperature during sleep and earlier increase in body temperature (NPL 3). Additionally, such poor sleep quality in the elderly has been reported to worsen conditions of patients with Alzheimer's disease and Parkinson's disease. Thus, improvements in reduced core body temperature and sleep quality in the elderly are major issues.

PTL 1 describes a body temperature regulating agent composed of γ-aminobutyric acid (GABA) as a body temperature regulating agent capable of suppressing increase in body temperature and easily reducing body temperature. According to PTL 1, GABA acts on parasympathetic nerves to suppress increases in blood pressure and heart rate. Additionally, GABA causes skin surface vasodilation, which increases skin blood flow, thereby promoting body heat loss on the skin surface. As a result, excessive increase in blood temperature is suppressed, which suppresses increase in body temperature in a deep part of the body.

In addition, according to PTL 1, when body temperature temporarily increases, γ-aminobutyric acid acts on the parasympathetic nerves, as in the above case, which causes mitigation of increased blood pressure and heart rate, increase in skin blood flow, and reduction in blood temperature, thereby promptly reducing increased body temperature. As a result, deep sleep is obtained.

PTL 2 describes a sleep improving agent including D-ribose as an active ingredient. PTL 2 shows the effect that continuously administering D-ribose at a dose of 350 to 3000 mg/kg of body weight per day promoted increases in non-REM sleep of mouse models of sleep disorder caused by imposed stress and suppressed reduction in REM sleep thereof. Note that PTL 2 also states that D-ribose hardly affected a circadian variation in core body temperature of mice without imposed stress.

On the other hand, Tongkat Ali is a plant that belongs to Simaroubaceae and is known as Nagae Casa (Japanese name); Eurycoma longifolia (Scientific name), and naturally grows mostly in lowland forests in Southeast Asia, such as Indonesia and Malaysia. Tongkat Ali is locally used as a material of traditional folk medicine, and, for example, PTL 3 to PTL 6 describe efficacies of extracts from roots of Tongkat Ali.

PTL 3 describes the use of a bioactive component of an extract from a root of Eurycoma longifolia in treatment for sexual dysfunction or male infertility. In addition, it is also described that there is literature reporting that a quassinoid extract extracted by chromatography from Eurycoma longifolia is potentially useful in treatment of cancer, ulcer, malaria, and fever.

PTL 4 describes a male function enhancer including a Tongkat Ali extract. It is also described that the Tongkat Ali extract includes eurycomanone as a primary component, and further includes 13α-epoxyeurycomanone, Eurycoma-lactone, 14,15β-dihydroxyklaineanone, organic acid, and other bioactive components, in which eurycomanone as the primary component has antimalarial effect.

PTL 5 describes a composition including a polar organic extract of Eurycoma longifolia. It is described that the extract has a percentage by weight of up to 5%, and quassinoids, coumarins, and glycosides, analogues, and derivatives thereof are included in the composition, the composition being used as a male sexual function improving agent.

PTL 6 states that an extract of Eurycoma longifolia enhances and/or stimulates an immune system/immune function to exhibit antiaging effect so as to protect the body from infectious disease and thereby reduce diseases due to weakened immune system (for example, disease states such as cancer and aging).

However, there is no literature describing a relationship between Tongkat Ali and core body temperature. It is thus needless to say that there is no literature stating or suggesting that Tongkat Ali reduces core body temperature. In other words, it is not known that Tongkat Ali has core body temperature reducing effect.

### CITATION LIST

### Patent Literature

PTL 1: JP 2007-204406 A
PTL 2: JP 2015-218119 A
PTL 3: JP 2004-521075 A
PTL 4: JP 2009-51765 A
PTL 5: JP 2010-500342 A
PTL 6: JP 2018-502079 A

### Non Patent Literature

NPL 1: The Journal of the Japanese Society of Balneology, Climatology and Physical Medicine, Vol. 78, No. 1, November 2014.
NPL 2: Aminosan “gurisin” (*1) sessyu niyori, nyumincyu no shinbutaion o teikasase, suimin no shitsu ⋅ suiminryo ga kaizensareru koto o hakken!—Presented at the 32^{nd} Meeting of the Japanese Society of Sleep Research—[online], Oct. 24, 2007, Ajinomoto Co., Inc., [Search on Jun. 8, 2018], Internet <URL: https://www.ajinomoto.com/jp/presscenter/press/detail/2007_10_24.html>
NPL 3: Shuichiro Shirakawa & Kunihiro Ichinose: Rojin no Senmo, Suimin Kakusei Rizumu Syogai to Sono Chiryo, “Seitai Rizumu to Seishin Shikkan” edited by Japanese Society of Biological Psychiatry, Gakkai Shuppan Center, pp 71-64 (1997).

### SUMMARY OF INVENTION

### Technical Problem

It is an object of the present invention to provide a novel core body temperature reducing agent.

### Solution to Problem

To achieve the above object, one aspect of the present invention provides a core body temperature reducing agent including an extract from Tongkat Ali as an active ingredient.

### Advantageous Effects of Invention

According to the present invention, a novel core body temperature reducing agent is provided.

### BRIEF DESCRIPTION OF DRAWINGS

FIG. 1 is a graph illustrating a result obtained by an experiment conducted in Example, which graph illustrates circadian variations in core body temperature of mice in a Tongkat Ali feeding group at the start of free feeding (0 wk) and 3 weeks (3 wk) and 4 weeks (4 wk), respectively, after the start thereof;

FIG. 2 is a graph illustrating a result obtained by the experiment conducted in Example, which graph illustrates circadian variations in core body temperature of mice in a control group at the start of free feeding (0 wk) and 3 weeks (3 wk) and 4 weeks (4 wk), respectively, after the start thereof;

FIG. 3 is a graph illustrating a result obtained by the experiment conducted in Example, which graph illustrates changes in the total daily amount of activity of the mice in each group at the start of free feeding (0 wk) and per week (1 wk to 4 wk) in the period up to 4 weeks after the start thereof;

FIG. 4 is a graph illustrating a result obtained by the experiment conducted in Example, which graph illustrates changes in feed intake of the mice in each group at 1 week before free feeding (−1 wk), at the start of free feeding (0 wk), and per week (1 wk to 4 wk) in the period up to 4 weeks after the start thereof;

FIG. 5 is a graph illustrating a result obtained by the experiment conducted in Example, which graph illustrates changes in body weight of the mice in each group at 1 week before free feeding (−1 wk), at the start of free feeding (0 wk), and per week (1 wk to 4 wk) in the period up to 4 weeks after the start thereof;

FIG. 6 is a graph illustrating a result obtained by the experiment conducted in Example, which graph illustrates changes in feed intake per g of body weight of the mice in each group at the start of free feeding (0 wk) and per week (1 wk to 4 wk) in the period up to 4 weeks after the start thereof; and

FIG. 7 is a graph illustrating a result obtained by the experiment conducted in Example, which graph illustrates changes in water intake of the mice in each group at 1 week before the start of free feeding (−1 wk), at the start of free feeding (0 wk), and per week (1 wk to 4 wk) in the period up to 4 weeks after the start thereof.

### DESCRIPTION OF EMBODIMENTS

### [Aspects of the Present Invention]

A first aspect of the present invention is a core body temperature reducing agent including an extract from Tongkat Ali (hereinafter referred to as “Tongkat Ali extract”) as an active ingredient. In the core body temperature reducing agent, the extract is preferably an extract from a root of Tongkat Ali, and is more preferably a hot water extract from a root of Tongkat Ali. The extract from Tongkat Ali may be powder or liquid.

The content of the Tongkat Ali extract with respect to the total amount of the core body temperature reducing agent is, for example, from 0.0001% by mass to 50% by mass, and preferably from 0.01% by mass to 30% by mass.

A second aspect of the present invention is a food product including the core body temperature reducing agent of the first aspect.

A third aspect of the present invention is a sleep improving agent including the core body temperature reducing agent of the first aspect.

### [Description of Structure of Each Aspect]

### <Tongkat Ali>

The type of Tongkat Ali (Eurycoma longifolia) used as a target for extraction is not limited, and any of yellow, red, and black types or any other type than those may be used. Additionally, a plurality of types of Tongkat Ali may be mixed together and used. The part of Tongkat Ali to be used is not limited, and, for example, may be root, bark, stem, or leaf. Among them, root is preferably used.

### <Method for Manufacturing Extract>

Pretreatment steps for extraction may be performed, such as a step of washing, drying, or freeze-drying Tongkat Ali collected as a plant and a step of grinding to obtain a uniform specimen.

As an extraction method, a known method can be employed. Examples of the known extraction method include water extraction, solvent extraction, steam extract, subcritical water extract, and supercritical carbon dioxide extraction.

Water extraction is a method of extracting an extract by immersing a target object in room temperature water or high temperature hot water. Instead of using only water, a liquid prepared by adding a small amount of an organic solvent such as ethanol to water may be used for extraction.

Solvent extraction is a method of extracting an extract from a target object using a polar or nonpolar organic solvent, in which an organic solvent aqueous solution may be used. Examples of the polar organic solvent include alcohol, acetone, and ethyl acetate. Examples of the nonpolar organic solvent include chloroform and toluene. Among them, from the viewpoints of safety to human body and handleability, aliphatic alcohols having 2 to 4 carbon atoms, such as ethanol, propanol, and butanol, are preferably used, and an ethanol aqueous solution is particularly preferable.

Water extraction is a preferable extraction method, and a more preferable extraction method is hot water extraction using only water. Extraction temperature is preferably 85° C. or higher, more preferably 90° C. or higher, and still more preferably 95° C. or higher.

### <Core Body Temperature and Reduction in Core Body Temperature>

Core body temperature means a temperature of a deep part in the body (for example, rectum, esophagus, heart, brain, or the like), and in the case of humans, core body temperature is usually a rectum temperature or an esophagus temperature.

Reduction in core body temperature means that the core body temperature of an individual becomes lower than a normal value. It is difficult to specifically determine, in a numerical range, how much the core body temperature is reduced, because there are species difference, individual difference, sexual difference, and differences depending on the amount of exercise and age. However, generally, in the case of experimental animals such as rats, core body temperature is reduced by about 0.5° C. to 3° C. less than a normal value. Additionally, in the case of humans as well, core body temperature is reduced by about 0.5° C. to 3° C. less than a normal value (in general, from 33.5° C. to 36.0° C. with respect to a normal body temperature of 36.5° C.)

### <Additives Included in Core Body Temperature Reducing Agent>

The core body temperature reducing agent of the first aspect may include other additives than the Tongkat Ali extract that are acceptable as pharmaceutical additives and food additives. Examples of such additives can be as follows, but not limited thereto:

Sugars such as lactose, glucose, and sucrose; starches such as corn starch and potato starch; cellulose and its derivatives, such as sodium carboxymethyl cellulose, ethyl cellulose, and cellulose acetate; cyclodextrin, dextrin, and the like; tragacanth powder; malt; gelatin; talc; stearic acid; magnesium stearate; calcium sulfate;

Vegetable oils such as corn oil, cotton seed oil, and olive oil; polyols such as propylene glycol, polypropylene glycol, glycerin, sorbitol, mannitol, and polyethylene glycol; phosphate buffer; cocoa butter; emulsifier; Vaseline, paraffine, beeswax, and emulsion base; and other non-toxic compatible materials;

Wetting agents and lubricants, such as magnesium stearate, as well as colorants, flavors, fragrances, emulsifiers, excipients, tableting agents, stabilizers, antioxidants, and preservatives.

### <Use Forms of Core Body Temperature Reducing Agent>

The core body temperature reducing agent of the first aspect can be used as a medicine, quasi-medicine, or food product for mammals, or can be used to manufacture them. The term “food product” as used herein encompasses food as a whole. It encompasses, besides ordinary food including so-called health food, food with health claims such as food for specified health uses and food with nutrient function claims defined in the Food with Health Claims System by the Japanese Ministry of Health, Labour and Welfare, supplements, and the like, and further encompasses livestock feed and pet food. Additionally, as dosage forms for the core body temperature reducing agent, there may be mentioned, not only oral administration, such as food intake, but also transdermal administration, transpulmonary administration, and transmucosal administration, and the like.

Forms of the medicine, quasi-medicine, and food can be any of solid, semi-solid, and liquid forms, and examples thereof include tablet form, pill form, capsule form, liquid form, syrup form, powder form, granular form, ointment form, and cream form. In addition, as forms for the transpulmonary administration, there may be mentioned spray form, steam form, minute powder form, and the like.

Examples of the transdermal administration include addition to massage oil or cream, addition to a bath agent, soap, shampoo, rinse, or in-shower face mask, addition to a material for a massage tool, and addition to an enzyme bath.

The food product of the second aspect is a food product including the core body temperature reducing agent of the first aspect, and as the forms thereof, there may be mentioned beverage and food products other than beverage.

Examples of the beverage include tea beverages, coffee beverages, milk beverages, fruit juice beverages, carbonated beverages, alcoholic beverages, soft drinks, and soups.

Examples of the food products other than beverage include bread, noodles, jelly food, various kinds of snacks, baked sweets, cakes, chocolates, chewing gums, candies, tablets, capsules, dairy products, frozen foods, convenience foods, supplements, other processed foods, seasonings, and materials thereof.

### <Intended Users of Core Body Temperature Reducing Agent>

The core body temperature reducing agent of the first aspect is applicable not only to humans but also to pets (pet animals), livestock, and the like, and applicable to mammals as a whole for medical purposes or non-medical purposes. Specifically, the core body temperature reducing agent is applicable to, besides humans, for example, dogs, cats, mice, rats, rabbits, bovine, horses, monkeys, and the like.

### <Sleep Improving Agent>

The expression “improvement in sleep content” means to obtain a state, such as easier falling asleep, improved insomnia, deep sleep, or feeling refreshed on awakening, and means improvements in the quality of sleep as a whole, such as promotion of natural sleep induction (promoted sleep onset) and improved sensation of deep sleep. More specifically, it means to enhance the level of sleep satisfaction of people who have sleep-related complaints, such as light sleep, feeling unrefreshed on awakening, and difficulty falling asleep, although not at the level requiring a sleeping pill or a sleeping inducer.

Improvement in sleep content can be evaluated by measuring electroencephalogram, electromyogram, electrocardiogram, body temperature, blood pressure, action (locomotion), and the like from an animal such as a mouse and examining the amount of sleep of the animal. Evaluation on the quality of sleep can be evaluated by a known method, and can be performed, for example, according to a standard scoring system (Rechtschaffen A. & Kales A., A manual of Standardized Terminology, Techniques and Scoring System for Sleep Stages of Human Subjects., Public Health Service: Washington D.C., 1968).

Specifically, a polysomnogram may be generated based on measurement data such as an electroencephalogram from the animal, and the state of the animal may be determined into respective stages: awakening, non-REM sleep, and REM sleep on the basis of the presence or absence of action (locomotion), amplitude of the electroencephalogram (δ wave), θ wave component ratio [θ/(δ+θ)] of the electroencephalogram, and the like per epoch (for example, 4 to 60 seconds) of the polysomnogram. The polysomnogram-based determination as to whether it is awakening or sleeping can be automatically made by a sleep analysis research software “SleepSign (registered trademark)” produced by Kissei Comtec Co., Ltd., or the like. As a result of the determination, a substance that has increased the stage of sleep can be evaluated as a substance having an effect of improving sleep quality and capable of improving sleep disorder.

A sleep improving agent of the third aspect includes the core body temperature reducing agent of the first aspect, and has, particularly, an effect of shortening sleep latency and an effect of extending non-REM sleep time. The sleep latency means a time required to fall asleep from being awake, and becomes an index of easy falling asleep. The sleep improving agent of the third aspect can be used for treatment of sleep disorders in which insomnia is a chief complaint. Examples of sleep disorders to which the sleep-improving agent of the third aspect is applicable include insomnia, initial insomnia (difficulty falling asleep), difficulty staying asleep, early morning awakening, deep sleep disorder, and inverted sleep cycle, but not limited thereto. The sleep improving agent is applicable to overall sleep disorders in which insomnia is the chief complaint.

### EXAMPLES

Hereinafter, Example of the present invention will be described. However, the present invention is not limited to Example given below. While Example below includes technologically preferable limitations in order to implement the present invention, the limitations are not essential requirements of the present invention.

<Preparation of Extract from Tongkat Ali>

After washing and air-drying a root of yellow Tongkat Ali, the root was crushed into pieces with an appropriate size. Then, 500 g of the crushed material was pulverized into powder by Labo Millser (Osaka Chemical Co., Ltd). The powder of Tongkat Ali obtained by the pulverization was added to 5 L of distilled water, and hot water extraction was performed at 90° C. for 1 hour. The obtained extraction solution was cooled down to 50° C. or lower, and then subjected to suction filtration using filter paper. The filtrate was frozen-dried to obtain 58.5 g of powder. The powder is a hot water extract from the root of Tongkat Ali.

### <Preparation of Feed Including Tongkat Ali Extract>

Mixed feed was prepared using the powder of Tongkat Ali extract obtained by the above-described method and feed AIN-93M obtained from Oriental Yeast Co. Ltd. Specifically, the Tongkat Ali extract was added to and mixed with the feed AIN-93M in such a manner that the content of the Tongkat Ali extract was 0.25% by mass, followed by tableting and drying with hot air to obtain a pellet of mixed feed. If a mouse (body weight: 25 g) is fed with 4 g of the mixed feed, it means that the feed intake of the mouse is 400 mg/kgBW of the test substance (the Tongkat Ali extract).

### <Experiment Using Mice>

Four each of mice (C57/BL6N-SLC, 10-week-old male mice) purchased from Japan SLC Co. Ltd., were housed in each one cage of an experimental plot and a control plot. The rearing environment was set as follows: light-on time point 8:00, light-off time point 20:00, and cage temperature 25° C. First, rearing was started using the feed AIN-93M as feed for all the mice.

Next, after a while from the start of the rearing, a body temperature/activity measuring device (“nano tag (registered trademark)” produced by Acos Co., Ltd.,) was placed near small intestinal spaces in the abdominal cavities of all the mice under inhalation anesthesia with 2.5% isoflurane. After the placement, a 2-week postoperative recovery period was provided. During the 2 weeks, the above-mentioned mixed feed was fed to all the mice in the cage of the experimental plot, whereas the AIN-93M was fed to all the mice in the cage of the control plot. For 4 weeks after that, the above-mentioned mixed feed was placed in the cage of the experimental plot to allow free feeding for all the mice in the experimental plot, whereas the AIN-93M was placed in the cage of the control plot to allow free feeding for all the mice in the control plot.

Then, immediately after passage of the 2-week postoperative recovery period (at the start of free feeding), and at 1, 2, 3, and 4 weeks, respectively, after the start thereof, core body temperature and the amount of activity of each mouse for 24 hours were measured by the body temperature/activity measuring device. The measurement was conducted every 5 minutes from 8:00 every Sunday morning to 8:00 on Monday morning, and each data was input to the body temperature/activity measuring device. After ending of the measurement, all the data was transferred and received from the body temperature/activity measuring device into a personal computer, and stored therein.

Additionally, every week from 1 week before the start of free feeding, body weights of all the mice were measured, and also, feed intake and water intake per gauge were measured.

### <Experimental Results>

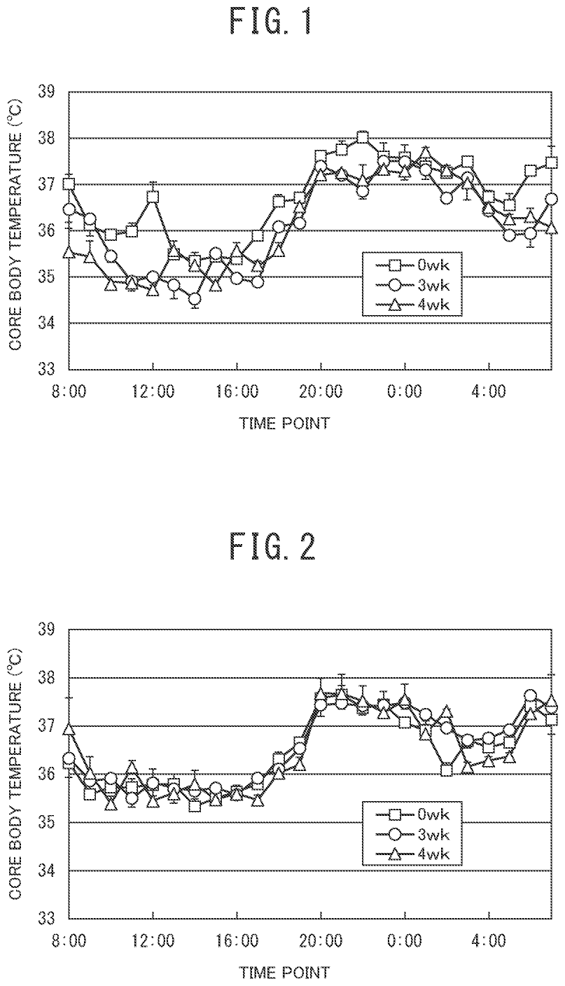

FIGS. 1 and 2 each illustrate a diurnal variation of core body temperature in the mice at the start of free feeding (0 wk), and 3 weeks (3 wk) and 4 weeks (4 wk), respectively, after the start of free feeding, in which FIG. 1 illustrates a result of the experimental plot, and FIG. 2 illustrates a result of the control plot. In graphs of FIGS. 1 and 2, plots of core body temperature at each time point indicate average values among the 4 individuals of each group in average values of the data (each data for 0 minute, 5 minutes, 10 minutes, and 15 minutes) measured every 5 minutes in each mouse.

As illustrated in FIG. 1, in the Tongkat Ali extract feeding group (the experimental plot), a clear difference was observed between a core body temperature variation in the mice at the start of free feeding (0 wk) and core body temperature variations in the mice at 3 weeks (3 wk) and 4 weeks (4 wk), respectively, after the start thereof. The core body temperatures of the mice at 3 weeks (3 wk) and 4 weeks (4 wk), respectively, after the start of free feeding were clearly lower than the core body temperatures of the mice at the start thereof (0 wk). The body temperature reduction was significant during rest period (light period), and even during activity period (dark period), a slight body temperature reduction was observed.

On the other hand, as illustrated in FIG. 2, the control group (the control plot) not fed with the Tongkat Ali extract exhibited no clear difference between a core body temperature variation in the mice at the start of free feeding (0 wk) and core body temperature variations in the mice at 3 weeks (3 wk) and 4 weeks (4 wk), respectively, after the start thereof.

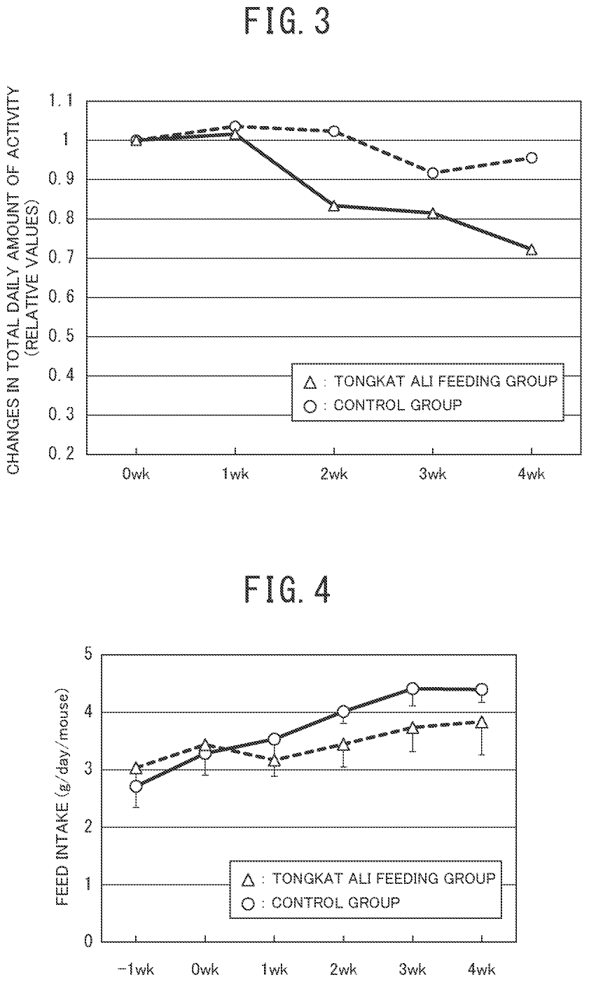

Additionally, FIG. 3 is a graph illustrating changes in the total daily amount of activity at the start of free feeding (0 wk) and per week (1 wk to 4 wk) in the period up to 4 weeks after the start thereof. The total daily amount of activity means a cumulative value of all data per day regarding the amount of activity measured every 5 minutes, and each plot on the total amount of activity at each time point in the graph of FIG. 3 indicates relative values calculated assuming that the total amount of activity at the start of free feeding (0 wk) is 1.

As illustrated in FIG. 3, the total daily amount of activity in the Tongkat Ali extract feeding group (the experimental plot) was observed to be slightly less than in the control group, which was, however, not noticeable.

FIG. 4 is a graph illustrating changes in feed intake in each group at 1 week before the start of free feeding (−1 wk), at the start of free feeding (0 wk), and per week (1 wk to 4 wk) in the period up to 4 weeks after the start thereof. Each plot of FIG. 4 indicates values obtained by dividing a total weekly feed intake of each group by the number of individuals and the number of days.

As illustrated in FIG. 4, in the Tongkat Ali extract feeding group, intake amount reduction was observed in the first week after the start of free feeding, but gradual recovery was observed from the second week thereafter.

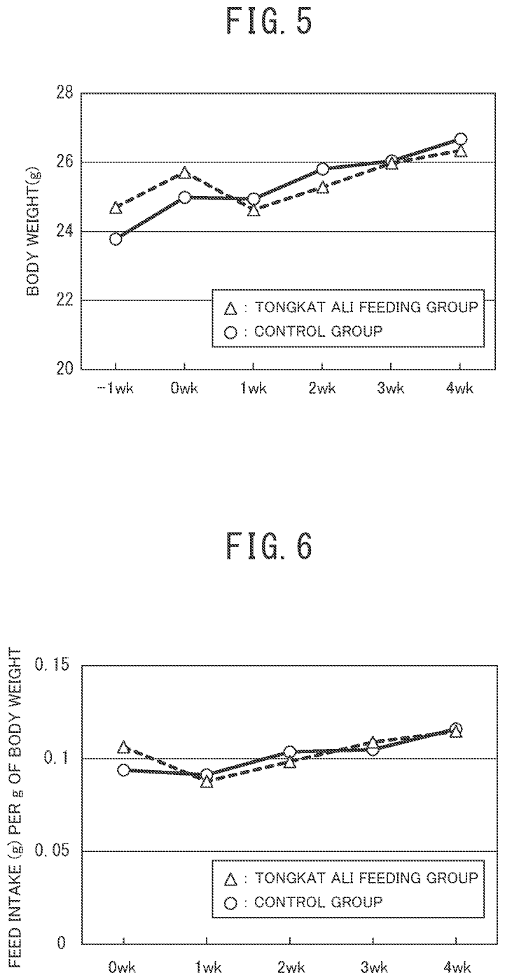

FIG. 5 is a graph illustrating changes in body weight of the mice in each group at 1 week before the start of free feeding (−1 wk), at the start of free feeding (0 wk), and per week (1 wk to 4 wk) in the period up to 4 weeks after the start thereof. Each plot of FIG. 5 indicates average values among the 4 individuals of each group.

As illustrated in FIG. 5, body weight in both group was reduced more or less in the first week after the start of free feeding. However, after that, the body weight was increased more moderately than changes expected from increased amounts of intake.

FIG. 6 is a graph illustrating changes in feed intake per g of body weight in each group at 1 week before the start of free feeding (−1 wk), at the start of free feeding (0 wk), and per week (1 wk to 4 wk) in the period up to 4 weeks after the start thereof.

As illustrated in FIG. 6, in the Tongkat Ali extract feeding group, feed intake per g of body weight reached around 99% of the control group from 1 week after the start of free feeding, and recovered to substantially the same amount as that at the start of free feeding in 4 weeks after the start thereof.

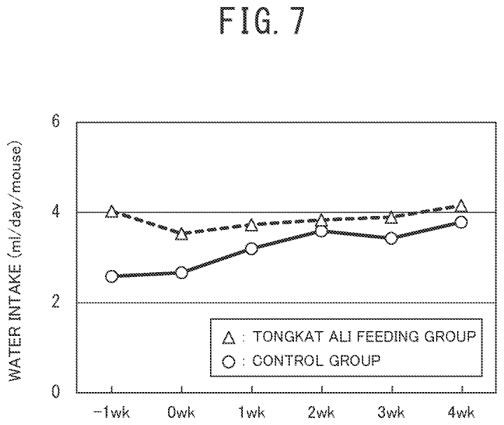

FIG. 7 is a graph illustrating changes in water intake of each group at 1 week before the start of free feeding (−1 wk), at the start of free feeding (0 wk), and per week (1 wk to 4 wk) in the period up to 4 weeks after the start thereof. Each plot of FIG. 7 indicates values obtained by dividing a total weekly water intake of each group by the number of individuals and the number of days.

As illustrated in FIG. 7, both group exhibited no significant changes in water intake from 1 week before the start of free feeding up to 4 weeks after the start thereof.

### CONCLUSION

The above experimental results show that as compared with the mice not fed with the Tongkat Ali extract, the mice fed with the Tongkat Ali extract have exhibited significant reduction in core body temperature, particularly, in core body temperature during light period (rest period). In addition, in the mice fed with the Tongkat Ali extract, slight reductions in feed intake and body weight were temporarily observed, but immediately recovered. Thus, it can be determined that the reduced core body temperature is not due to the reduced feed intake but due to effect of the Tongkat Ali extract.

Furthermore, regarding daily activity rate, particularly, during the activity period, there was no significant difference between the mice fed with the Tongkat Ali extract and the mice not fed with the Tongkat Ali extract. This shows that the Tongkat Ali extract serves to efficiently reduce core body temperature, particularly, during the rest period without significantly affecting activity during the activity period.

Those described hereinabove have shown that the Tongkat Ali extract can be an active ingredient of a core body temperature reducing agent, and that a food product including the Tongkat Ali extract is effective in reducing core body temperature. It has been also shown that since reduced core body temperature in sleep latency improves sleep content, the Tongkat Ali extract can be an active ingredient of a sleep improving agent.

Note that the core body temperature reducing agent of the first aspect and the sleep improving agent of the third aspect including the plant-derived Tongkat Ali extract as the active ingredient are much safer than medicines, and therefore can be taken in over a long period of time.

Furthermore, the core body temperature reducing agent of the first aspect is expected to be used as a prevention agent against heat stroke during sleep in summer, which often occurs in the elderly, and used as a heat stroke-preventing agent for livestock animals (such as cattle) reared in livestock industries and the like.

1. A core body temperature reducing agent comprising an extract from Tongkat Ali as an active ingredient.

2. The core body temperature reducing agent according to claim 1, wherein the extract is an extract from a root of Tongkat Ali.

3. The core body temperature reducing agent according to claim 2, wherein the extract is a hot water extract.

4. The core body temperature reducing agent according to claim 1, wherein core body temperature reducing effect is more significant during rest period than activity period.

5. (canceled)

6. (canceled)
